(19) United States
(12) Patent Application Publication
(10) Pub. No.: US 2009/0238890 A1
(43) Pub. Date: Sep. 24, 2009

(54) CONTINUOUS SPRAY-CAPTURE PRODUCTION SYSTEM

(75) Inventors: John Piechocki, Redwood City, CA (US); David J. Kyle, Catonsville, MD (US)

Correspondence Address: MOORE & VAN ALLEN PLLC, P.O. BOX 13706, Research Triangle Park, NC, 27709, US

(73) Assignee: ADVANCED BIONUTRITION CORPORATION, Columbia, MD (US)

(21) Appl. No.: 12/160,497

(22) PCT Filed: Jan. 16, 2007
(86) PCT No.: PCT/US07/01126

§ 371 Date: Dec. 9, 2008

Related U.S. Application Data
(60) Provisional application No. 60/758,792, filed on Jan. 13, 2006.

(51) Int. Cl. A61K 9/14

(52) U.S. Cl. 424/501

(57) ABSTRACT

The disclosure relates to novel microencapsulation processes based on the use of high viscosity fluids (e.g., gelatinized starch and alginate), which are mixed and then sprayed using a much gentler hydraulic pressure and, preferably gas-based atomization into a crosslinking solution (e.g. of calcium chloride). To improve the efficiency of the system, the process can be performed in a continuous mode rather than by a conventional batch process. This involves continuous or intermittent harvest of the microparticles collected in the capture vessel followed by amendment and recycling of the CaCl₂ solution and its redeployment into the capture vessel. The process allows production of microencapsulated probiotic bacteria without major losses in viability, thereby providing a useful and efficient new manufacturing method for the stabilization of probiotic bacteria prior to their introduction into functional foods.

### BACKGROUND OF THE DISCLOSURE

The disclosure relates generally to the fields of packaging and delivery of bacteria.

Probiotic bacteria are bacteria that colonize the gastrointestinal tract of animals or man and provide beneficial effects to the host organism. The health benefits of food products containing probiotic bacteria (e.g., yogurt, fermented milk products) have been known for thousands of years in traditional medicine. However, a very high percentage of probiotic bacteria are destroyed by the stomach before they can reach the small intestine where they have their beneficial effect.

Harel et al (U.S. patent application Ser. No. 10/534,090) have shown that if probiotic bacteria can be encapsulated in a matrix that provides gastric protection, then much lower doses need be used in the functional food. However, the manufacturing process described was only a batch process and although effective, there are economic disadvantages to operations run as batch processes relative to running in a continuous process. Other manufacturing challenges of providing stabilized, viable bacteria in a food product outside the dairy case, at high enough concentrations to provide functional benefits to the consumer, have not been solved. Overcoming these challenges would open up major new “functional food” markets for this country's manufacturing base, and provide new products with significant health benefits to consumers as a whole. The present invention provides a solution to the continuous delivery of viable probiotic bacteria in a functional food by a novel method of microencapsulation of the probiotic bacteria into particles of 100-250 μm in diameter.

Polymer matrices such as those proposed by Harel (U.S. patent application Ser. No. 10/534,090) generally consist of different types of starch and/or other polymers such as poly(vinylpyrrolidone), poly(vinylalcohol), poly(ethylene oxide), cellulose (and cellulose derivatives), silicone and poly(hydroxyethylmethacrylate) (see also U.S. Pat. No. 6,190,591 for examples of suitable materials). A combination of starch and emulsifier has also been envisioned as a method for delivery of materials to foods (see U.S. Pat. No. 6,017,388).

Cross-linked and non-digestible starch has been proposed to enhance the growth of probiotic bacteria in a prebiotic fashion (see U.S. Pat. No. 6,348,452). Harel has proposed a combination of starch and alginate, the latter of which is cross linked by calcium ions by spraying the mixture into a bath containing a combination of 5% calcium chloride and 1% sodium chloride using air pressure and atomizing the material using a paint sprayer (U.S. patent application Ser. No. 10/534,090). This was a batch process where the microparticles so produced were filtered following atomization and then stored. Although the encapsulated materials with the demonstrated composition so produced were useful as a gastric preservation method, the efficiency of the overall process was limited, there was a certain amount of probiotic cell damage using the air powered atomization, many probiotic cells are very sensitive to chloride damage, and the throughput was relatively slow. The present invention provides a solution to all of these processing problems.

### BRIEF SUMMARY OF THE DISCLOSURE

This novel microencapsulation process developed by the inventors is based on the use of very high viscosity fluids (gelatinized starch and alginate), which are mixed and then sprayed using a much gentler hydraulic pressure and air-based atomization into a cross-linking solution of calcium chloride for low concentration with no supplemental sodium chloride. In order to improve the efficiency of the system the inventors further developed the process to allow this production process to take place in a continuous mode rather than by a conventional batch process. This involved the continuous harvest of the microparticles collected in the capture vessel followed by amendment and recycling of the CaCl₂ solution and its redeployment into the capture vessel. The inventors discovered that the concentration of Ca²⁺ ions in the capture vessel is critical and needs to be maintained for the effective cross-linking of alginate microgels, while any buildup of chloride levels can be toxic to the bacteria or corrosive to the equipment. The invention described herein further teaches how to maintain the Ca²⁺ and Cl⁻ levels using selective addition of Ca²⁺ and removal of Cl⁻ levels from the process stream prior to its reintroduction into the capture vessel. The inventors also discovered that surprisingly, the starch alginate mixture also absorbed chloride ion as well as used the Ca²⁺ for cross linking. Finally, the process developed allows for the production of the microencapsulated probiotic bacteria without major losses in viability, thereby providing a useful and efficient new manufacturing method for the stabilization of probiotic bacteria prior to their introduction into functional foods.

### BRIEF SUMMARY OF THE SEVERAL VIEWS OF THE DRAWINGS

FIG. 1, consisting of FIGS. 1A and 1B, is a pair of graphs that illustrate changes in the Ca²⁺ and Cl⁻ levels during the production process in “low volume” experimental run 1 without CaCl₂ amendment (FIG. 1A), and experimental run 2 with CaCl₂ amendment (FIG. 1B).

FIG. 2, consisting of FIGS. 2A and 2B, is a pair of graphs that illustrate changes in the Ca²⁺ and Cl⁻ levels during the production process at full volume (200 L) without Cl⁻ removal (FIG. 2A), and with Cl⁻ removal by ion exchange (FIG. 2B).

FIG. 3 consists of FIGS. 3A and 3B. FIG. 3A is a flow diagram, and FIG. 3B is an image of a unit operation, of the Cl⁻ reduction system using ion exchange resin.

FIG. 4, consisting of FIGS. 4A and 4B, is a pair of graphs that illustrate changes in the Ca²⁺ and Cl⁻ levels during the full volume production process (200 L) including probiotic bacteria and without Cl⁻ removal (FIG. 4A), or with Cl⁻ removal using ion exchange (FIG. 4B).

FIG. 5, consisting of FIGS. 5A, 5B, 5C, and 5D, is a quartet of graphs that illustrate changes in pH of process tank as a function of time during hydrogel formation without probiotic bacteria (FIGS. 5A and 5B), and with probiotic bacteria (FIGS. 5C and 5D), and impact of Cl-removal using ion exchange resin process (FIGS. 5B and 5D).

### DETAILED DESCRIPTION

The disclosure relates to encapsulation of bacteria, such as probiotic bacteria, and other materials in microbeads suitable for ingestion by animals and use in production of food materials, for example.

Production of Microbeads.

High viscosity compositions generally cannot be pumped with much efficiency through narrow orifices to produce a fine spray such as in spray drying. One can use, however, a spray jet nozzle that provided hydraulic pressure to move the material and then use a post-nozzle air vortex to disrupt the viscous fluid of from 1,000 cps to 25,000 cps into finer particles. One such nozzle is the ¼ JHU-SS Automatic Air Atomizing Nozzle produced by Spraying Systems, but other similar jet nozzles can be used as well. Any high pressure pumping system can be used such as the AutoJet system manufactured by Spraying Systems Inc (Chicago, Ill.).

A high viscosity, alginate-containing composition such as described by Harel (U.S. patent application Ser. No. 10/534,090) can be prepared and Probiotic bacteria such as, but not limited to species of Lactobacillus, Bifidobacteria, Enterococcus, Streptococcus, and Pseudoalteromonas is then added to the high viscosity, alginate-containing material. This material is well mixed in a mixing tank and the resulting material is pumped using a hydraulic liquid pump at pressures from 30 psig to 100 psig through a fluid jet nozzle such as, but not limited to (¼ JHU-SS) (Spraying Systems, Chicago, Ill.). Air, nitrogen, carbon dioxide, or any inert gas at pressures of from 30 psig to 60 psig is also pumped into the jet nozzle so that the atomization of the high viscosity material can take place outside the jet nozzle. The jet nozzle is located from 10 to 1,000 cm above the surface of a capture liquid comprising a cross linking material such as calcium chloride at a concentration of from 2.5 to g/L to 20.0 g/L. The particles so produced can range in size from 10 to 1000 microns based on the distance from the nozzle to the capture liquid surface. A preferred embodiment results in the production of particles from 50 to 250 microns in diameter.

In order to minimize the aerosols not hitting the surface of the capture liquid or bouncing off the surface of the capture liquid as series of oversprayers can be used to provide a “liquid cover” of the same or similar composition as the capture liquid. Such oversprayers will also provide “channeling” of the microparticles and initiate cross-linking even prior to contact of the microbead with the surface of the capture liquid.

Process Recycling.

A recycle loop is then coupled to the harvest system of the process tank such that the filtrate from the harvest sieves, which removed the product, could be pumped back into the process tank through the oversprayers. The system of “oversprayers” simultaneously act as an aerosol containment system for the main process tank and they continuously rinse the sidewalls.

Using a composition of from 0.1% to 3% alginate (a preferred embodiment would be 0.75% to 1.5% alginate), and from 0.5% to 5% hydrated starch (a preferred embodiment would be 1% to 3% hydrated starch matrix) a mixture can be prepared for the formation of microparticles. Because of its high viscosity, the blending of this mixture into a smooth consistency requires a powerful high shear mixer. The blended standard mixture is referred to throughout this document as “A1,” can be loaded into a batch tank and pumped through the jet nozzle into a capture tank.

The newly formed product out is simultaneously pumped out of the process tank and this process stream can be fed directly to a harvesting device such as but not limited to filter screens (e.g., Liquitex separator). The filtered product can be collected at one screen outlet, while the filtrate is collected at another outlet and pumped back into the process tank using a bifurcated line that allows control of the volume being returned through the oversprayers, or through a surge line. Prior to the return of the process stream to the capture tank, the Ca²⁺, Cl⁻ and H⁺ ion concentrations can be monitored and the process stream can be amended to maintain a Ca²⁺, Cl⁻ and H⁺ ion concentration within predefined limits. This amendment can be through the addition of Ca²⁺ in the form of, but not limited to, calcium chloride, calcium sulfate or calcium carbonate, the removal of chloride by ion selective membranes or ion exchange resins, and the addition of protons by titration with acids such as, but not limited to sulfuric acid, nitric acid, and hydrochloric acid.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

### Example 1

### Preparation of Microparticles Using a Spray Capture Recycle System

For all test runs described in this report, the 1% alginate, 2% hydrated starch matrix composition was first prepared according to a standard recipe. Because of its high viscosity (ca. 1,400 cp), the blending of this mixture into a smooth consistency required a powerful high shear mixer. The blended standard mixture is referred to throughout this report as “A1,” and was loaded into the batch tank up to its maximum capacity of about 100 kg. The A1 mixture was then pumped through the jet nozzle at a flow rate of 0.267 gal/min (ca. 1 kg/min) using a fluid pump controlled at a fluid pressure of 25 PSIG. Formation of the microparticles at the jet nozzle also requires airflow, which was controlled with an air pressure of 50 PSIG, The product was then captured in a 0.4 m³ (100-gallon) process tank in a bath containing CaCl₂.

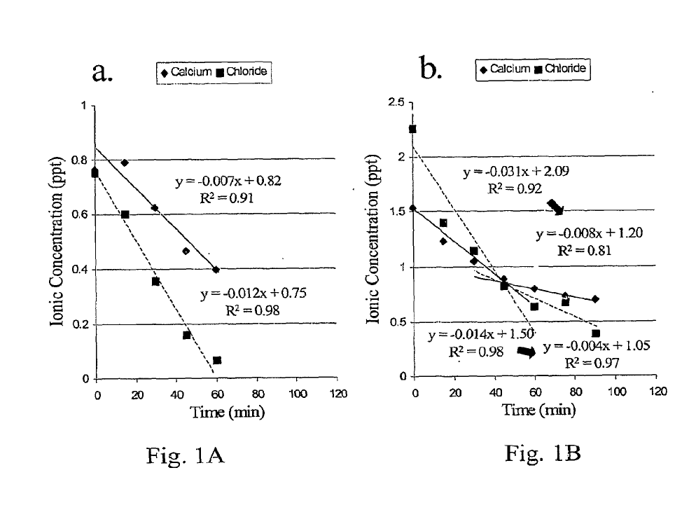

The recycling system was designed to pump the newly formed product out of the process tank at a flow rate of about 12 L/minute (180 gal/hr). This process stream was fed directly to a Liquitex separator fitted with two sets of screens (25 μm and 250 μm) (FIG. 1). The filtered product was collected at the screen outlets, and the filtrate was collected in a 50 L recycling tank equipped with level sensors. The recycle tank was outfitted with a central bottom drain and, under control of level sensors, a pump was activated and the filtrate was pumped back into the process tank through a bifurcated line that allows control of the volume being returned through the oversprayers, or through a surge line. Once operational, the system was easy to balance so that the filtrate level in the recycle tank remained stable and the oversprayers were constantly working. The system could operate in this recycle mode almost indefinitely.

The recycle tank was the location of “in-line” calcium ion (Ca²⁺), chloride ion (Cl⁻), and pH (H⁺) probes. Pasco ion selective electrodes and Explorer GLX data logging meters were used for the in-line monitoring of the Ca²⁺, Cl⁻ and H⁺ ion concentrations. Because of the potential for fouling of the electrodes by the jet nozzle, the in-line probes were not placed directly in the process tank as originally planned. These are robust electrodes and exhibited a linear response in the ion concentrations used in this process. The probes were calibrated before initiation of each of the experimental runs and, in some runs, discreet samples were taken and calorimetric assays used to confirm the various ion levels recorded by the in-line probes.

### Example 2

### Ca²⁺ and Cl⁻ Ions in the Process Tank in Recycle Mode

Control of the calcium and chloride ion levels in the process tank is critical for two reasons: 1) free Ca²⁺ ions are required to cross-link the liquid alginate to form a hydrogel particle; and 2) excessively high Cl⁻ levels were found to be injurious to the probiotic bacteria encapsulated in the hydrogel. With the recycle loop in place, the effect of the overall process on the Ca²⁺ and Cl⁻ levels in the process tank was determined.

The system as described in Example 1 was used with a liquid volume of the process stream held to a minimum (60 L) and a low CaCl₂ starting concentration was used in order to establish the magnitude of changes in the Ca²⁺ and Cl⁻ levels in response to the continuous production of an alginate hydrogel. The batch tank was filled to its maximum capacity of 100 kg of liquid matrix A1, and the process and recycle tanks were charged with at total of 60 L of 0.25% CaCl₂ solution. Process throughput was set to 0.267 gal/hr of the A1 mixture, and measured to be 1.04 kg/min by collecting and weighing 100% of the output from the nozzle over a 60 second period. Recycle volume flow was 12 L/min (80 gal/min) resulting in one complete change of the process tank approximately every 5 minutes. With no amendment to the CaCl₂ content in the process stream, the Ca²⁺ level dropped at a linear rate of about 7 ppm/min (FIG. 1A). Unexpectedly, the Cl⁻ levels also dropped at a rate of about 12 ppm/min. The operation was terminated after 45 minutes, at which time the A1 mixture was only weakly cross-linked or not cross-linked at all and a viscous liquid was clogging the harvest screens. Within the first 45 minutes the Ca²⁺ ion concentration in the system had dropped to 0.396 ppt (equivalent to 0.12% CaCl₂), which established the lowest effective concentration of calcium ions usable in this system.

Using the same minimal volumes in the production unit, a second experimental run was undertaken, but this time the starting Ca²⁺ ion concentration was doubled to 1.54 ppt (0.46% CaCl₂) and further supplemented by the addition of 500 mL of a solution of 7% CaCl₂ at 45, 60, and 75 minutes into the run. In this case the Ca²⁺ ion concentration did not fall below 0.7 ppt (0.21% CaCl₂) (FIG. 1B) and the entire 100 kg of A1 material was converted into hydrogel particles and collected by the separator within 60 minutes. Initial throughput was determined early in the run and again found to be 1.04 kg/min. The rate of drop in the Ca²⁺ ion concentration was significantly slowed from 14 to 4 ppm/min once the amendment was initiated with the additional of CaCl₂ (i.e., after 45 min). Unexpectedly, the rate of drop of the Cl⁻ ion concentrations was similarly slowed from 31 to 8 ppm/min by the amendment. In both experimental runs using a minimal amount of process liquid, the drop in the Cl⁻ ion concentration suggests that both calcium and chloride were being taken up by the hydrogel matrix at a ratio of approximately 1:2.

### Example 3

### Controlling of Ca²⁺ and Cl⁻ Levels During Continuous Operation

It was initially anticipated that the Ca²⁺ levels in the process liquid would drop at a rate predicted by the uptake of Ca²⁺ used for the cross linking of the alginate hydrogel, and that the Cl⁻ levels would remain constant. As a result of the amendment of the process liquid with additional CaCl₂, the Cl⁻ levels were predicted to rise. However, in the experiments of Example 2, it was discovered that the Cl⁻ levels were not remaining constant as the Ca²⁺ levels dropped, nor were they increasing as more CaCl₂ was added to the system. To ensure that this was not simply a phenomenon observed as a consequence of using such small quantities of process liquid in theses initial experiments, these experiments were repeated using 200 L (50 gallons) of process liquid in the system.

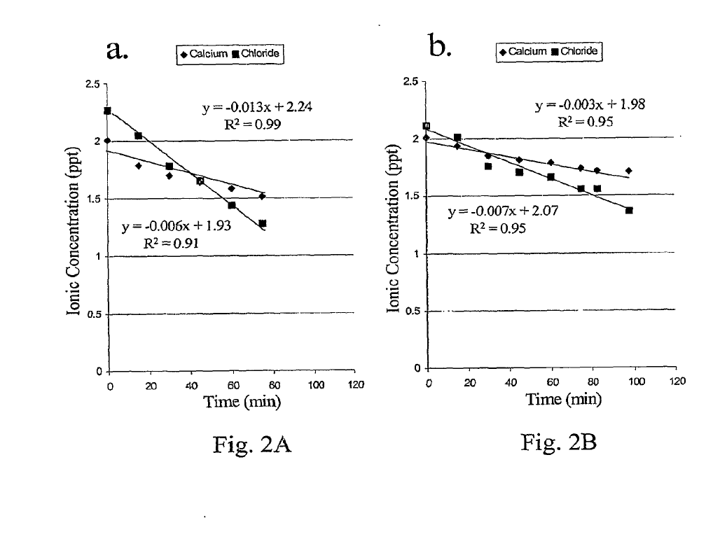

Based on the rate of Ca²⁺ depletion in the smaller scale experiments, the amount of supplemental CaCl₂ to be added was established. The A1 flow through the jet nozzle remained the same as in earlier experimental runs and was measured to be 1.06 kg of A1 per minute. At the same throughput rate, the Ca²⁺ and Cl⁻ depletion rates should be the same as in the previous runs even though the process liquid volume was increased. Consequently the CaCl₂ amendment rate was initially set to be the same as that in the small volume run of Example 2 (i.e., 500 mL of 7% solution every 15 min). The measured rates of Ca²⁺ depletion (6 ppm/min) and Cl⁻ depletion (13 ppm/min) were similar to those of the small-scale run except that the depletion rates were more linear throughout the run (FIG. 2A) as the CaCl₂ supplementation was started immediately, rather than after 45 minutes as in Example 2.

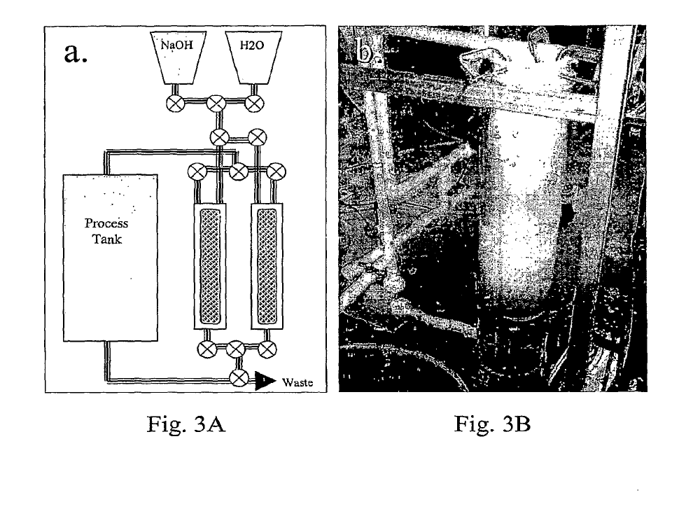

Consistent with the low volume runs of Example 2, the rate of Ca²⁺ depletion was about one-half the rate of Cl⁻ depletion, suggesting the uptake ratio of one Ca²⁺ atom for every two Cl⁻ atoms. This is consistent with a stoichiometric uptake of CaCl₂ by the hydrogel. Nevertheless, we developed a process for the reduction of accumulating Cl⁻ ion that involved a passage of a small volume of the process liquid (20 L) over an ion exchange resin (3 kg) to remove excess Cl⁻. This was followed by the re-addition of the Cl⁻ depleted process liquid to the process tank and the recharging of the ion exchange resin. Although this process was tested in a batch mode with a single ion exchange tank, it could be converted to a continuous operation using two deionizing tanks where Cl⁻ is being removed using the first tank while the resin is being recharged in the second tank as shown in the flow diagram in FIG. 3. The two tanks could then be cycled at any frequency required by the process. Using this system, a second large volume experimental run was completed using the parameters of the first run of this Example 3, but with the removal of Cl⁻ ion using the ion exchange process. Every 30 minutes throughout the run 10% of process liquid (20 L) was removed and mixed with 3 kg of anion exchange resin (Dowex Marathon A) for 15 minutes and then returned to the process tank. The ion exchange resin was subsequently recharged with 0.1 N NaOH, followed by extensive rinsing until the pH had returned to between 8 and 9. In addition to the Cl⁻ removal, the rate of CaCl₂ amendment was increased to 750 mL 7% CaCl₂/15 min in order to further reduce the rate of Ca²⁺ depletion. Depletion of Ca²⁺ under this new regimen was reduced to only 3 ppm/min and the Cl⁻ depletion rate was reduced to 7 ppm/min (FIG. 2B). Even though this new procedure would specifically eliminate accumulating Cl⁻ ion, the depletion rate was still in the ratio of two Cl⁻ ions for every Ca²⁺, questioning the need to implement this additional amendment step. As these slopes approach zero (i.e., the overall depletion rates approach zero), steady state conditions are obtained and the system could theoretically run indefinitely. On the basis of these performance data, we have concluded that the ion exchange system could be incorporated into the overall operation as a “safety valve” for the fine-tuning of Cl⁻ ion concentration, but it would likely not need to be run continuously.

### Example 4

### Full Scale Run with Probiotic Bacteria

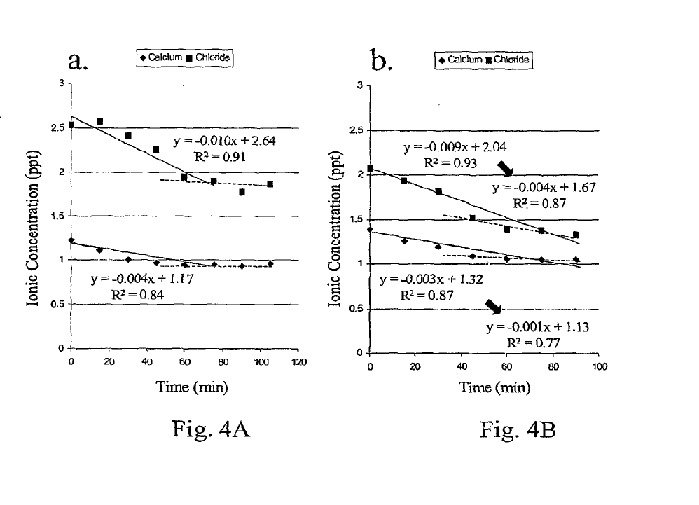

Hydrogels containing the probiotic bacterium Lactobacillus rhamnosus were prepared using the conditions established in Example 3, a flow throughput measured at 1.0 kg/min, and a CaCl₂ amendment rate of 600 mL of 7% CaCl₂ every 15 minutes for the first 75 minutes and 1000 mL every 15 min for the remainder of the run. For the first 80 minutes, the Ca²⁺ depletion rate was 4 ppm/min and the Cl⁻ depletion rate was 10 ppm/min (FIG. 4A). When the supplementation rate was increased to 1000 mL/15 min, both Ca²⁺ and Cl⁻ ion concentrations leveled out, or even appeared to increase slightly. However, the inclusion of the probiotic bacteria did not appear to change the fluid flow dynamics, nor the Ca²⁺ and Cl⁻ uptake rates in the system.

In an attempt to better focus the CaCl₂ amendment levels, a final experimental run was undertaken using A1 mixed with the probiotic bacteria, a CaCl₂ amendment of 750 mL/15 min, and with the ion exchange resin process to control Cl⁻ levels at 30, 60 and 90 minutes into the nm (FIG. 41B). The throughput on this last run was measured at 1.04 kg/min, demonstrating remarkable consistency in the jet nozzle and pumping system. Although the Ca²⁺ ion levels fell at a rate of 3 ppm/min and Cl⁻ dropped at a rate of 9 ppm/min over the entire experiment, the Ca²⁺ and Cl⁻ drop during the last hour of operation was reduced to 1 ppm/min and 4 ppm/min, respectively.

Samples were taken at various process steps and locations throughout both of these experiments, immediately chilled on wet ice, transferred to the laboratory, and prepared for live cell counts. Since the principal concern with respect to cell viability was in the high shear environment of the jet nozzle, samples were taken at the feed tank (prior to the jet nozzle) and at the outlet into the harvester (after the formation of the hydrogel particles). Live cell counts indicated that there was little damage to the viability of the bacteria by the spray capture process (loss of about 40%), nor as a consequence of the 90 minutes residence time in the feed tank (Table 1). The apparent low level of recovery at the initial time point may simply have been due the fact that the system had not yet reached an equilibrium state. Particles from the harvest tanks (large particles and small particles) both had about the same bacterial count on a dry weight basis. This was not unexpected, as the A1 material was uniformly mixed with the bacteria in the batch tank before spraying and the bacterial concentration in the hydrogel should not be affected by particle size. There was a small amount of hydrogel material that flowed into the recycle tank that accounted for less than 0.1% of the total mass of the A1 after 90 minutes. The lower bacterial count in these very small (<10 μm) particles may reflect a surface area to volume limitation on loading, or the possibility that the bacteria are better protected if in the internal space of the particle rather than exposed on the surface. The lack of viable bacteria in the recycle tank supernatant would support this view that the viability of the bacteria is enhanced by being embedded in the hydrogel matrix.

Table 1 summarizes live cell counts of Lactobacillus rhamnosus before and after encapsulation process (a) and resident in the harvest tanks (large and small particles) vs. the recycle tank. Note that 99% of the hydrogel was collected from the harvest tanks.

TABLE 1

| Time | Feed Tank | Harvest Stream | % |
|---|---|---|---|
| (min) | (×10⁹ cfu/gdw) | (×10⁹ cfu/gdw) | Recovery |
| 0 | 28.0 | 9.2 | 33% |
| 30 | 33.7 | 25.2 | 75% |
| 60 | 35.4 | 20.9 | 59% |
| 90 | 29.2 | 18.1 | 62% |
| Mean | 31.6 | 18.4 | 58% |

| | Viability |
|---|---|
| | (×10⁹ cfu/gdw) |
| Large particles | 27.1 |
| Small particles | 16.9 |
| Recylcle tank particles | 1.2 |
| Recycle tank supernatent | 0.01 |

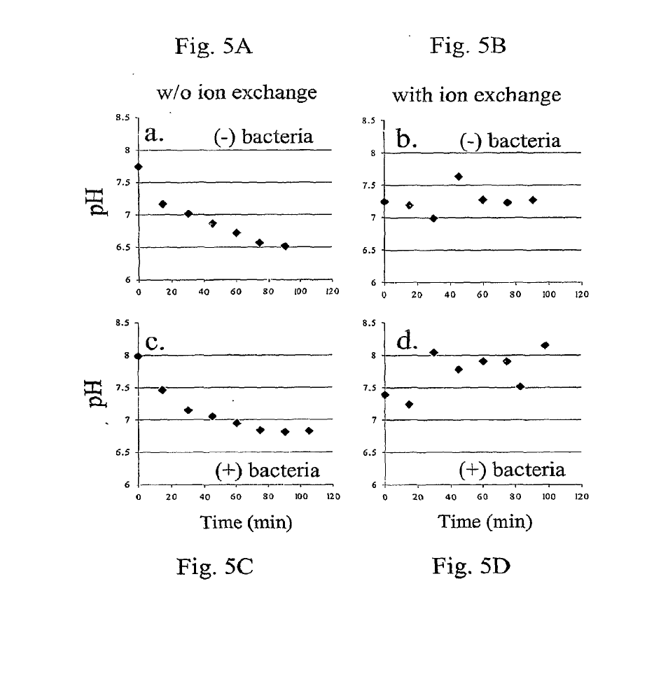

Throughout the course of all the experiments pH was monitored in the process tanks. No attempt was made at this time to control the pH and it was generally seen to drift down about 1-1.5 units over 90 minutes. The presence of probiotic bacteria in the A1 hydrogel mixture did not seem to affect the course of the downward pH drift (FIG. 5). However, when the ion exchange resin procedure was employed in an attempt to reduce Cl⁻ ion build up, there was a significant leveling effect on the pH drift and the pH was held between 7.0 and 8.0. This may have been through the introduction of a small amount of residual base associated with the recharging of the ion exchange resin.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subj ect matter described herein. The appended claims include all such embodiments and equivalent variations.

1. A process for the production of cross-linked microparticles, the process comprising atomizing an alginate-containing liquid having a viscosity not less than 1,000 centipoise and capturing the atomized liquid in a second liquid containing calcium ions, whereby the atomized liquid droplets become cross-linked to form the microparticles.

2. The process as in claim 1, the second liquid is a liquid in which CaCl₂ is dissolved.

3. The process as in claim 2, wherein the concentration of CaCl₂ in the second liquid is maintained between 2.5 and 20.0 g/L.

4. The process as in claim 2, wherein the concentration of calcium ions in the second liquid is maintained between 0.72 and 5.74 g/L.

5. The process as in claim 2, wherein the concentration of chloride ions is maintained at a concentration of below 14.3 g/L.

6. The process as in claim 1, wherein the alginate-containing liquid includes live bacteria.

7. The process as in claim 6, wherein the bacteria are probiotic bacteria.

8. The process as in claim 7, wherein the probiotic bacteria include bacteria selected from the group of genera consisting of Lactobacillus, Bifidobacteria, Streptococcus, Enterobacteria, and Pseudoalteromonas.

9. The process as in claim 1, wherein the diameter of most of the microparticles formed are in the range from 10 to 1000 microns

10. The process as in claim 1, wherein diameter of most of the microparticles formed are in the range from 50 to 250 microns

11. The process as in claim 1, wherein the alginate-containing liquid includes starch.

12. The process as in claim 1, wherein the alginate-containing liquid has a viscosity not greater than 25,000 centipoise.

13. The process as in claim 1, wherein the alginate-containing liquid is atomized by passing it at a hydraulic pressure not less than 30 psig through an atomization nozzle.

14. The process as in claim 14, wherein a gas is passed through the atomization nozzle together with the alginate-containing liquid.

15. The process in claim 1, wherein the droplets of atomized liquid are permitted to settle under gravity into a container containing the second liquid.
